SEQUENCE LISTING (18 sequences)

SEQ ID NO: 1 - RNA, 20 nt, Synthetic construct
  source 1..20: mol_type = other RNA
tctttgcagccagtctctcg

SEQ ID NO: 2 - RNA, 20 nt, Synthetic construct
  source 1..20: mol_type = other RNA
tctcgcgggcaaatacgtca

SEQ ID NO: 3 - DNA, 26 bp, Synthetic construct
  source 1..26: mol_type = other DNA
tttcactgtaatgatttttcaagagc

SEQ ID NO: 4 - DNA, 26 bp, Synthetic construct
  source 1..26: mol_type = other DNA
cagacaaataaccttctaagcaaggg

SEQ ID NO: 5 - RNA, 20 nt, Synthetic construct
  source 1..20: mol_type = other RNA
catatgactagctggccaca

SEQ ID NO: 6 - DNA, 25 bp, Synthetic construct
  source 1..25: mol_type = other DNA
tatggccgaagcatcttatgaatgt

SEQ ID NO: 7 - DNA, 25 bp, Synthetic construct
  source 1..25: mol_type = other DNA
cacaactctttccacacaccaacaa

SEQ ID NO: 8 - RNA, 1406 nt, Macrobrachium rosenbergii
  source 1..1406: mol_type = mRNA
cgatgacatttcagcggctgtaagcactgttcccattggccgaaagtctgcattgtggggccgagagagaggcgacgggccaatgggcggcggcgtgacaaaagctctcgtcaccggacggactcggcggagtgatatatgacgtcactactcgcactacgccccttcaagttgaacatcctcatcggagtcagtcactaatagtcagtctgtgttccctagtgatcagaactttggtgaattggatttacgatggcttcaggagtccaagtatcggatacagtaatagagtacttccaacaaataaagaagggaaagatgtataggtacataatcttccacattaaagatgacaaggtcattgatgttgaaacgactggagatagagacgctagttatgatgactacttaaactacctggtaaatttggggcctgaacagtgccgttatggcatctatgattttgaatacgatcatcagtgccagggaacgacagattctacgaggaagcagaaacttttcctgatgtcttggtgcccagatactgccaagatcaaaaagaagatgctgtattcatcaagctttgatgcccttaaacaatcattggaaggcataggaaagtacatacaggctacagatatggccgaagcatcttatgaatgtgttttggacaagatgcgctctactgacagaacctaagttagtgatgggaggcccctctgttctggagagcccccgctgtatttgaggggtccctttgtctctaccgccctgtggccagctagtcatatgtgggccacaatcatgctatccacgcagcatgaagagaactttgtttaattattttttattcacaatttgttggtgtgtggaaagagttgtggggaaacagtgtttcaccagaagaaaggttaggcttgatacaaatgccatgtgggagagtggaccggctttaaagttaggacaaatagtcttgacattaaagctggtcccaagtgatgtgggagaggcagtgtttcatgcattgctacagagttggcagaggagagttagtattaaatataaatgctcttccatacgtattttgtcccattgtatgtgatcttggatctttcacatcttcatagatataataagctttatataaacagtatagccatttaccatttattggctccaaaaattattttattaactgctcaaaatccttaattgcatcagcttcattatgtgaatcatgaatttagtttaaaaagggatttttttatgatttaaatgtaggatcaacttgtcagaataaatttctttttaatgttggtgattttatttcatcttgcctttggttaaagaaactgcagttgaaaatcgtcaactggatgtgattatgatcatggatattaagtttt

SEQ ID NO: 9 - protein, 148 aa, Macrobrachium rosenbergii
MASGVQVSDTVIEYFQQIKKGKMYRYIIFHIKDDKVIDVETTGDRDASYDDYLNYLVNLGPEQCRYGIYDFEYDHQCQGTTDSTRKQKLFLMSWCPDTAKIKKKMLYSSSFDALKQSLEGIGKYIQATDMAEASYECVLDKMRSTDRT

SEQ ID NO: 10 - DNA, 44 bp, Macrobrachium rosenbergii
  source 1..44: mol_type = genomic DNA
ctttgtctctaccgccctgtggccagctagttagtcatatgtgg

SEQ ID NO: 11 - DNA, 75 bp, Macrobrachium rosenbergii
  source 1..75: mol_type = genomic DNA
agtttgagagaactcattaccctgacgtatttgcccgcgagagactggctgcaaagataggcctgccggaagcta

SEQ ID NO: 12 - DNA, 69 bp, Macrobrachium rosenbergii
  source 1..69: mol_type = other DNA
agtttgagagaactcattacccatttgcccgcgagagactggctgcaaagataggcctgccggaagcta

SEQ ID NO: 13 - DNA, 69 bp, Macrobrachium rosenbergii
  source 1..69: mol_type = other DNA
agtttgagagaactcattaccctgttgcccgcgagagactggctgcaaagataggcctgccggaagcta

SEQ ID NO: 14 - DNA, 73 bp, Macrobrachium rosenbergii
  source 1..73: mol_type = other DNA
agtttgagagaactcattaccctgatatttgcccgcgagagactggctgcaaagataggcctgccggaagcta

SEQ ID NO: 15 - DNA, 56 bp, Macrobrachium rosenbergii
  source 1..56: mol_type = other DNA
agtttgagagaactcattacagagactggctgcaaagataggcctgccggaagcta

SEQ ID NO: 16 - DNA, 69 bp, Macrobrachium rosenbergii
  source 1..69: mol_type = other DNA
agtttgagagaactcattacgtatttgcccgcgagagactggctgcaaagataggcctgccggaagcta

SEQ ID NO: 17 - DNA, 57 bp, Macrobrachium rosenbergii
  source 1..57: mol_type = other DNA
agtttgagagaactcattacgagagactggctgcaaagataggcctgccggaagcta

SEQ ID NO: 18 - DNA, 73 bp, Macrobrachium rosenbergii
  source 1..73: mol_type = other DNA
agtttgagagaactcattaccctggtatttgcccgcgagagactggctgcaaagataggcctgccggaagcta

(19) United States
(12) Patent Application Publication
(10) Pub. No.: US 2024/0381857 A1
(43) Pub. Date: Nov. 21, 2024

(54) GENOME EDITING OF GROWTH, DISEASE RESISTANCE/IMMUNITY AND CUTICLE COLOR IN CRUSTACEANS

(71) Applicants: B. G. Negev Technologies and Applications Ltd., at Ben-Gurion University, Beer Sheva (IL); COLORS FARM LTD., Moshav Hatzeva (IL)

(72) Inventors: Amir SAGI, Beer Sheva (IL); Rivka MANOR, Beer Sheva (IL); Eli AFLALO, Beer Sheva (IL); Ran EPSTEEN, Moshav Hatzeva (IL); Marcia PIMENTA LEIBOWITZ, Sapir (IL); Rivki OFIR, Moshav Hatzeva (IL)

(73) Assignees: B. G. Negev Technologies and Applications Ltd., at Ben-Gurion University, Beer Sheva (IL); COLORS FARM LTD., Moshav Hatzeva (IL)

(21) Appl. No.: 18/669,129

(22) Filed: May 20, 2024

Related U.S. Application Data
(60) Provisional application No. 63/467,322, filed on May 18, 2023.

(51) Int. Cl. A01K 67/033; C12N 5/07; C12N 9/22; C12N 15/11

(52) U.S. Cl. CPC A01K 67/0338; C12N 5/0601; C12N 9/22; C12N 15/111; A01K 2217/075; A01K 2227/70; C12N 2310/20

(57) ABSTRACT

The present invention provides a method for editing the genome of at least one cell of a crustacean embryo, the method including contacting a crustacean embryo or a cell thereof with an effective amount of a gene editing agent. Further provided is a crustacean including at least one cell comprising an edited genome, obtained according to the method of the invention.

### CROSS-REFERENCE TO RELATED APPLICATIONS

This application claims the benefit of priority of U.S. Provisional Patent Application No. 63/467,322, titled “GENOME EDITING OF GROWTH, DISEASE RESISTANCE/IMMUNITY AND CUTICLE COLOR IN CRUSTACEANS”, filed 18 May 2022, the contents of which is incorporated herein by reference in its entirety.

### REFERENCE TO AN ELECTRONIC SEQUENCE LISTING

The contents of the electronic sequence listing (BGU-P-0136-US.xml; size: 18,930 bytes; and date of creation: May 18, 2024) is herein incorporated by reference in its entirety.

### FIELD OF INVENTION

The present invention is in the field of molecular biology, and specifically is directed to, inter alia, a method for editing the genome of a crustacean, including, cells and/or embryo thereof.

### BACKGROUND OF THE INVENTION

The giant freshwater prawn Macrobrachium rosenbergii (Decapoda, Crustacca) is one of the most important species in crustacean aquaculture, but its production volumes have not increased significantly in the past decade. Despite several attempts to improve yields through classical selective breeding and efforts to adjust M. rosenbergii aquaculture to industrial-sized indoor systems through monosex culture, there has been no marked improvement to date in yields per area. It is thus evident that other measures of genetic improvement are needed for enhancing yields. A promising minimal-intervention way forward (in contrast to radical genomic modifications through genetic engineering) may lie in a single basepair knock out (KO) through clustered regularly interspaced short palindromic repeats (CRISPR) gene editing.

Particularly when used in concert with a Cas protein (the CRISPR/Cas system), CRISPR editing has become a prominent technique that has found application in the generation of edited organisms and in gene function research. With advances in this system, genome editing of unconventional and non-model research animals is now possible. Many applications were theoretically suggested and some have now been implemented in practice in certain fields. As part of this trend, the advantages of the CRISPR/Cas implementation have been studied in aquaculture, one of the fastest growing commercial food sectors, and promising results have already been obtained for genetically edited fish. Similarly, in cultured crustaceans, which comprise a significant and fast-growing sector of the aquaculture industry, initial experiments indicate exciting future prospects but also a pressing need to establish appropriate tools.

In efforts to develop such tools, one of the primary considerations is that genome editing in animals requires an intervention at early stages of development. This consideration also holds for crustaceans, whose early developmental stages follow the standard oviparous embryonic and larval development path common to most arthropods. In M. rosenbergii, embryonic development is characterized by a distinctive phenomenon in which the first cell divisions occur only after the first two nuclear divisions. Thus, in the current study, it was this phenomenon that directed the platform for CRISPR editing to early-stage embryos at exactly the same stage(s). Since this ‘constraint’ is difficult to realize experimentally, the inventors also used an alternative platform based on primary cell cultures, which offers the advantage of higher throughput experimentation vis-à-vis whole animal editing.

To establish and calibrate the CRISPR system in M. rosenbergii for both the above platforms, the inventors looked for a highly expressed gene with a supposedly favorable chromatin architecture to serve as reference. Actin-depolymerizing factor (ADF)/cofilin, which was first discovered in porcine brain, appeared to be an appropriate candidate gene for the study. Its translated product is bound to actin and it is involved in actin filament dynamics, indicating that it is essential for cukaryotic cells. Although no studies have been performed on crustacean cofilin, homologs of cofilin have been mentioned in transcriptomic and proteomic studies in several crustacean species, which suggests that such a gene does indeed exist in the M. rosenbergii genome.

Following a study of cofilin designed to demonstrate the possibility of Cas9 KO in M. rosenbergii, proof of concept for editing with an early clear phenotype was needed. For this purpose, the non-lethal Paired box protein 6 (Pax6), a highly conserved transcription factor that plays a crucial role in early eye development, was selected. Previous studies have shown that expression of Pax6 leads to a clear eye development phenotype at an early embryonic stage. In a study of the decapod Exopalaemon carinicauda revealed two Pax6 homologs containing a homeodomain (Hox) domain, that was used as a target for CRISPR.

### SUMMARY OF THE INVENTION

In some embodiments, the present invention is based, at least in part, on the findings that the CRISPR system was found to be a reliable and effective platform for genomic editing of primary cell cultures and embryos derived from a decapod crustacean.

According to the first aspect, there is provided a method for editing the genome of at least one cell of a crustacean embryo, the method comprising contacting a crustacean embryo at the 1-cell to 8 cell stage with an effective amount of a gene editing agent, thereby editing the genome of at least one cell of a crustacean embryo.

According to another aspect, there is provided a crustacean comprising at least one cell comprising an edited genome, obtained according to the method of the invention.

In some embodiments, the crustacean embryo is at the 1-cell to 4-cell stage.

In some embodiments, the contacting is injecting.

In some embodiments, the injecting is pneumatically injecting.

In some embodiments, the pneumatically injecting is under pressure of 15 to 25 psi.

In some embodiments, the pneumatically injecting is for a period of 150-250 msec.

In some embodiments, the agent is a clustered regularly interspaced short palindromic repeat (CRISPR) system.

In some embodiments, the CRISPR system comprises: (i) a CRISPR-associated endonuclease 9 (Cas9) protein or a nucleic acid sequence encoding thereof; and (ii) a guide RNA (gRNA) targeting at least one protospacer within the genome of the at least one cell of a crustacean embryo.

In some embodiments, the CRISPR system comprises: (i) a Cas9 protein; and (ii) a gRNA targeting at least one protospacer within the genome of the at least one cell of a crustacean embryo.

In some embodiments, the method further comprises a step before the contacting, comprising mixing the Cas9 protein and the gRNA.

In some embodiments, the editing comprises deleting at least one nucleotide from the genome of the at least one cell of a crustacean embryo compared to a non-edited control genome.

In some embodiments, the deleting comprises introducing any one of a non-synonymous mutation, a nonsense mutation, and a frameshift, in at least one gene of the edited genome such that a transcript transcribed therefrom, a protein product thereof, or both, is at least partially inactivated.

In some embodiments, the embryo comprises a plurality of embryos, and wherein 10-100% of the plurality of embryos being contacted with the gene editing agent comprises at least one cell comprising an edited genome.

In some embodiments, the 20-80% of the plurality of embryos being contacted with the gene editing agent comprises at least one cell comprising an edited genome.

In some embodiments, the 50-100% of the plurality of embryos being contacted with the gene editing agent survive the contacting.

In some embodiments, the 80-100% of the plurality of embryos being contacted with the gene editing agent survive the contacting.

In some embodiments, the method further comprises a step comprising culturing the crustacean embryo comprising the at least one genome edited cell to a developmental stage selected from the group consisting of: larva, post larva, juvenile, adult, and any combination thereof. In some embodiments, the crustacean is a decapod crustacean.

In some embodiments, the decapod crustacean is Macrobrachium rosenbergii.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1B include micrographs and schemes showing early embryonic development of M. rosenbergii. (1A) Early nuclear and cell divisions of M. rosenbergii embryos. The dark areas in the eggs are the nuclei, as shown in the schematic representation below the photographs. Two initial divisions of the nucleus occur prior to cell formation at 4 and 6 hours post fertilization (HPF). At 8 HPF, the egg divides into 4 cells (rather than splitting into two), and one hour later divides again into 16 cells. Subsequent divisions occur at 1.5-h intervals. Scale bars are 100 μm. (1B) Eyed eggs and cells in culture from eyed embryos. Left—Eyed embryo: Eye development starts at 9-10 days post fertilization, and these embryos contain a large number of cells that are suitable for extraction. Middle and right—Primary cell cultures of extracted cells containing various types of cells. All scale bars arc 100 μm.

FIGS. 2A-2C include a graph, a scheme, and sequences showing Mr-cofilin expression pattern, genomic and transcript structure in M. rosenbergii. (2A) Mr-cofilin expression pattern during embryogenesis constantly shows high expression in both sexes. Bars represent means and standard errors of normalized read counts. For day 1, n=5 three biological repeats from male embryos and two from female embryos, and for all subsequent days n=6, three repeats of each sex. (2B) The genomic structure of Mr-cofilin comprises five exons (dark squares) and four introns (gray lines), totaling 12,302 bp. (2C) Mature mRNA (SEQ ID NO: 8) comprises 1,406 bp with an open reading frame (ORF) of 477 bp and long 5′ and 3′ untranslated regions (UTRs); the guide RNA is situated at the 3′ UTR 769 bp. The putative translated protein (SEQ ID NO: 9) is 148 amino acids long and contains actin de-polymerization factor (ADF)/cofilin-like domains. Start and stop codons are shaded in gray.

FIG. 3 includes a graph showing comparison of Cas9 mRNA versus ribonucleoprotein (RNP) in embryos or embryonic cells in primary cell culture for Mr-cofilin editing. Injections into 1-cell to 4-cell embryos and nucleofections to primary cell culture with either Cas9 mRNA (gray) or RNP (empty boxes) with Mr-cofilin gRNA. For embryos, RNP editing efficiency averaged 73.4±18.2% (n=6), and mRNA editing efficiency, 24.7±14.8% (n=5). For cell culture successful editing was observed only in the RNP group, at a rate 42±1% (n=8). Bars represent means of editing efficiency according to inference of CRISPR edits (ICE) results±standard error.

FIGS. 4A-4B include graphs showing calibration and efficiency of RNP injection into M. rosenbergii embryos. (4A) RNP injections at different early stages of M. rosenbergii embryo cell divisions. Injections of Cas9 RNP, with Mr-cofilin gRNA, were administered to embryos from fertilization (1-cell) until the 32-cell stage. Number of cells refers to cell divisions; n=6 for all stages. Bars represent means of editing effect according to ICE analysis of Sanger sequencing±standard error with ‘a’ and ‘b’ signifying a significant difference between the groups (one way ANOVA, p=0.02). (4B) Calibration of Cas9 RNP conjugated with Mr-cofilin gRNA injection volume into M. rosenbergii 1-cell to 4-cell embryos. Bars represent ICE analysis of Sanger sequencing deletions (percentage), means±standard error. For all volumes n=6.

FIGS. 5A-5B include box plots showing editing efficiencies under optimal conditions using gRNAs from various genes in embryos and primary cultured M. rosenbergii embryonic cells. Results of next generation sequencing showing editing efficiencies of various targets, including (5A) 23 different genes and 80 guides for 40 samples in embryos (white) and 69 in primary cell culture (gray); and (5B) 3 Mr-cofilin repeats in embryos (white), and 7 Mr-cofilin repeats in in primary cell culture (gray). The results are given for 1-cell to 4-cell embryos injected with 670 pL of RNP solution and cell nucleofection of several different broods.

FIGS. 6A-6B include sequences, schemes, and graphs showing editing patterns in M. rosenbergii embryos versus embryonic primary cell culture. (6A) Crispresso2 representation of editing pattern in M. rosenbergii embryos and primary cell culture with Mr-cofilin sgRNA. Mr-cofilin sequence is presented at the bottom of each block with indication of guide location at the bottom (gray line). White dotted line signifies the cut site, and black bars represent deletions (percentage) at each position. Numbers represent unedited cases (percentage) at a given position. (6B) Graphic representation of the distribution of averaged deletions per position in embryos (left) and cell culture (right), n=3 for both, with significant differences (p<0.001) between the two distribution patterns using a permutations test. Black dashed lines signify cut sites, and bars represent standard error. All values upstream of the cut site in primary cell culture were 0%. The presented sequence in (A) and (B) is:

(SEQ ID NO: 10)
CTTTGTCTCTACCGCCCTGTGGCCAGCTAGTTAGTCATATGTGG.

FIGS. 7A-7D include micrographs, a graph, and a multiple sequence alignment showing phenotypic proof of concept of editing through eye development in M. rosenbergii embryos and larvae. (7A) Representative images of 15-day-old M. rosenbergii embryos injected with different gRNAs: left—Hox4, middle—Hox3 and right—wild type. (7B) A graph showing eye area (mm²) of 15-day old embryos for each gRNA; bars represent means±standard errors of eye area and ‘a’ and ‘b’ represent significant differences between the groups. (7C) Representative pictures of stage-1 larvae: top—Hox4-injected larva and bottom—wild-type larva. (7D) ICE analysis of Sanger sequencing results of samples for the Hox domain of MrPax6: top-reference sequence; bottom—Hox4-injected larva Sanger sequencing. ‘Indel’ indicates the number of deleted bases found in each sequence, ‘Contribution’ indicates the percentage of amplicons with similar sequences, and ‘Sequence’ indicates a window around the cut site. All scale bars are 100 μm; gRNA sequences are presented in Table 1.

FIG. 8 includes a non-limiting scheme showing a representation of editing platforms in M. rosenbergii. Gravid females are collected after fertilization (left). One to four-cell embryos were taken for direct Cas9 RNPs injection (top). Ten to twelve-day old eyed-embryos were used for cell extraction followed by primary cell culture (bottom) and subjected to electroporation in the presence of Cas9 RNPs. The RNP Cas9 system (right) penetrates the cell in both platforms and cleaves the DNA according to the gRNA sequence inducing or leading to a double strand brake.

### DETAILED DESCRIPTION

The present invention, in some embodiments, provides a method for editing the genome of a crustacean cell. In some embodiments, the cell is a cell of an embryo. In some embodiments, the cell is obtained or derived from an embryo. In some embodiments, the embryo comprises the cell. In some embodiments, the cell is in the form of an embryo. In some embodiments, the cell comprises one or more cells. In some embodiments, the cell comprises at least one cell. In some embodiments, the cell comprises a plurality of cells. In some embodiments, the crustacean is a decapod crustacean.

### Method

According to the first aspect, there is provided a method for editing the genome of at least one cell of a crustacean. In some embodiments, the cell comprises a cell of an embryo.

In some embodiments, the comprising contacting a crustacean embryo with an effective amount of a gene editing agent.

In some embodiments, an embryo comprises an embryo being at the 1-cell to 2-cell stage, 1-cell to 4-cell stage, 1-cell to 8-cell stage, 1-cell to 16-cell stage, 1-cell to 32-cell stage, 2-cell to 4-cell stage, 2-cell to 8-cell stage, 2-cell to 16-cell stage, 2-cell to 32-cell stage, 4-cell to 8-cell stage, 4-cell to 16-cell stage, 4-cell to 32-cell stage, 8-cell to 16-cell stage, 8-cell to 32-cell stage, or 16-cell to 32-cell stage. Each possibility represents a separate embodiment of the invention.

In some embodiments, the crustacean embryo is at the 1-cell to 4-cell stage.

In some embodiments, contacting comprises administering. In some embodiments, contacting comprises injecting. In some embodiments, injecting comprises pneumatically injecting.

As used herein, the expressions “pneumatically injecting” or “pneumatic injection” refer to any means, devices, and methods which utilize and/or are based on high pressure so as to inject an agent of interest, e.g., gene editing agent, to a target, e.g., a cell.

Means for pneumatic injections are common and would be apparent to one of ordinary skill in the art, such as exemplified herein.

In some embodiments, pneumatically injecting is under pressure of 10 to 30 psi, 10 to 25 psi, 15 to 30 psi, 15 to 25 psi, 15 to 25 psi, 16 to 24 psi, 17 to 23 psi, 18 to 22 psi, or 19 to 21 psi. Each possibility represents a separate embodiment of the invention.

In some embodiments, injecting is at a volume of 10 to 100 pL, 50 to 200 pL, 150 to 300 pL, 200 to 800 pL, 500 pL to 1.5 μL, 50 pL to 2 μL, 70 pL to 500 pL, or 80 pL to 1.5 μL. Each possibility represents a separate embodiment of the invention.

A person of ordinary skill in the art would acknowledge that pneumatic injection is under pressure and for a defined period of time, e.g., pulse injection, such as of milliseconds (msec).

In some embodiments, the agent is a programmable engineered nuclease (PEN).

The term “programmable engineered nuclease (PEN)” as used herein, refers to a synthetic enzyme that cuts specific DNA sequences, derived from natural occurring nucleases involved in DNA repair of double strand DNA lesions, and enables direct genome editing.

In some embodiments, PEN used by the methods of the invention may be any one of a clustered regularly interspaced short palindromic repeat (CRISPR) class I or class II system.

In some embodiments, the PEN used by the method of the invention may be a CRISPR Class 2 system. In some embodiments, such class 2 system may be a CRISPR type II system. In some embodiments, PEN comprises CRISPR type II system.

In some embodiments, the agent is a clustered regularly interspaced short palindromic repeat (CRISPR) system. In some embodiments, a CRISPR system comprises or is CRISPR system II.

In some embodiments, a CRISPR system comprises: (i) a CRISPR-associated endonuclease 9 (Cas9) protein, a nucleic acid sequence encoding thereof, or both; and (ii) a guide RNA (gRNA).

In some embodiments, a CRISPR system comprises: (i) a Cas9 protein; and (ii) a gRNA.

In some embodiments, the gRNA targets at least one protospacer within the genome of at least one cell of a crustacean embryo.

In some embodiments, the method of the invention further comprises a step comprising mixing a Cas9 protein and a gRNA. In some embodiments, the mixing step (e.g., of a Cas9 protein and a gRNA) is before or preceding the contacting (step). In some embodiments, the method of the invention further comprises a step before or preceding the contacting, comprising mixing a Cas9 protein and a gRNA.

As used herein, “CRISPR” or “CRISP arrays” also known as SPIDRs (Spacer Interspersed Direct Repeats) constitute a family of recently described DNA loci that are usually specific to a particular bacterial species. The CRISPR array is a distinct class of interspersed short sequence repeats (SSRs) that were first recognized in E. coli. In subsequent years, similar CRISPR arrays were found in Mycobacterium tuberculosis, Haloferax mediterranei, Methanocaldococcus jannaschii, Thermotoga maritima and other bacteria and archaea. It should be understood that the invention contemplates the use of any of the known CRISPR systems, particularly and of the CRISPR systems disclosed herein. The CRISPR-Cas system, targets DNA molecules based on short homologous DNA sequences, called spacers that exist between repeats. These spacers guide CRISPR-associated (Cas) proteins to matching (and/or complementary) sequences within the foreign DNA, called proto-spacers, which are subsequently cleaved. The spacers can be rationally designed to target any DNA sequence. Moreover, this recognition clement may be designed separately to recognize and target any desired target. With respect to CRISPR systems, as will be recognized by those skilled in the art, the structure of a naturally occurring CRISPR locus includes a number of short repeating sequences generally referred to as “repeats”. The repeats occur in clusters and are usually regularly spaced by unique intervening sequences referred to as “spacers.” Typically, CRISPR repeats vary from about 24 to 47 base pair (bp) in length and are partially palindromic. The spacers are located between two repeats and typically each spacer has unique sequences that are from about 20 or less to 72 or more bp in length. In some embodiments the CRISPR spacers used in the sequence encoding at least one gRNA of the methods and kits of the invention comprise between 10 to 75 nucleotides (nt) each. In some embodiments, the gRNA comprises at least: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or any vale and range therebetween. Each possibility represents a separate embodiment of the invention. In some embodiments, the gRNA comprises 70 to 150 nt. In some embodiments, the spacers comprise 20 to 35 nucleotides. In addition to at least one repeat and at least one spacer, a CRISPR locus also includes a leader sequence and optionally, a sequence encoding at least one tracrRNA. The leader sequence typically is an AT-rich sequence of up to 550 bp directly adjoining the 5′ end of the first repeat.

The term “CRISPR type II” system refers to a bacterial immune system that has been modified for genome engineering. It should be appreciated however that other genome engineering approaches, like zinc finger nucleases (ZFNs) or transcription-activator-like effector nucleases (TALENs) that relay upon the use of customizable DNA-binding protein nucleases that require design and generation of specific nuclease-pair for every genomic target may be also applicable herein. CRISPR-Cas systems fall into two classes. Class 1 systems use a complex of multiple Cas proteins to degrade foreign nucleic acids. Class 2 systems use a single large Cas protein for the same purpose. More specifically, class 1 may be divided into types I, III, and IV and class 2 may be divided into types II, V, and VI.

The type II CRISPR-Cas systems include the ‘HNH’-type system (Streptococcus-like; also known as the Nmeni subtype, for Neisseria meningitidis serogroup A str. Z2491, or CASS4), in which Cas9 is sufficient for generating crRNA and cleaving the target DNA, in addition to the ubiquitous Cas1 and Cas2. Cas9 contains at least two nuclease domains, a RuvC-like nuclease domain near the amino terminus and the HNH (or McrA-like) nuclease domain in the middle of the protein, but the function of these domains remains to be elucidated. However, as the HNH nuclease domain is abundant in restriction enzymes and possesses endonuclease activity responsible for target cleavage.

Type II systems cleave the pre-crRNA through an unusual mechanism that involves duplex formation between a tracrRNA and part of the repeat in the pre-crRNA; the first cleavage in the pre-crRNA processing pathway subsequently occurs in this repeat region. Still further, it should be noted that type II system comprise at least one of Cas9, Cas1, Cas2 csn2, and Cas4 genes. It should be appreciated that any type II CRISPR-Cas systems may be applicable in the present invention, specifically, any one of type II-A or B.

In some embodiments, the nucleic acid sequence encoding a Cas protein used in the method of the invention may be at least one Cas gene of type II CRISPR system (either typeII-A or typeII-B). In some embodiments, a nucleic acid encoding a Cas protein comprising a Cas gene of type II CRISPR system used by the method of the invention is the Cas9 gene. It should be appreciated that such a system may further comprise at least one of Cas1, Cas2, csn2 and Cas4 genes.

In some embodiments, a Cas protein consists or comprise a Cas9 protein. Double-stranded DNA (dsDNA) cleavage by Cas9 is a hallmark of “type II CRISPR-Cas” immune systems. The CRISPR-associated protein Cas9 is an RNA-guided DNA endonuclease that uses RNA:DNA complementarity to identify target sites for sequence-specific double stranded DNA (dsDNA) cleavage, creating the double strand brakes (DSBs) required for the HDR that results in the integration of the reporter gene into the specific target sequence, for example, a specific target within the avian gender chromosome Z. The targeted DNA sequences are specified by the CRISPR array, which is a series of about 30 to 40 bp spacers separated by short palindromic repeats. The array is transcribed as a pre-crRNA and is processed into shorter crRNAs that associate with the Cas protein complex to target complementary DNA sequences known as proto-spacers. These proto-spacer targets must also have an additional neighboring sequence known as a proto-spacer adjacent motif (PAM) that is required for target recognition. After binding, a Cas protein complex serves as a DNA endonuclease to cut both strands at the target and subsequent DNA degradation occurs via exonuclease activity.

CRISPR type II system as used herein requires the inclusion of two essential components: a “guide” RNA (gRNA) and a non-specific CRISPR-associated endonuclease (Cas9). The gRNA is a short synthetic RNA composed of a “scaffold” sequence necessary for Cas9-binding and about 20 nucleotide long “spacer” or “targeting” sequence which defines the genomic target to be modified. Thus, one can change the genomic target of Cas9 by simply changing the targeting sequence present in the gRNA. Guide RNA (gRNA), as used herein refers to a synthetic fusion of the endogenous bacterial crRNA and tracrRNA, providing both targeting specificity and scaffolding/binding ability for Cas9 nuclease. Also referred to as “single guide RNA” or “sgRNA”. CRISPR was originally employed to “knock-out” target genes in various cell types and organisms, but modifications to the Cas9 enzyme have extended the application of CRISPR to “knock-in” target genes, selectively activate or repress target genes, purify specific regions of DNA, and even image DNA in live cells using fluorescence microscopy. Furthermore, the case of generating gRNAs makes CRISPR one of the most scalable genome editing technologies and has been recently utilized for genome-wide screens.

In some embodiments, CRISPR type II system used for the method disclosed herein comprises CRISPR-associated endonuclease 9 (Cas9) and a gRNA.

In some embodiments, editing comprises deleting, inserting, or both. In some embodiments, editing comprises altering or modifying/changing a nucleic acid sequence of a genome of a crustacean, an embryo thereof, or a cell thereof.

In some embodiments, editing comprises any one of knock-in, knock-out, and both.

In some embodiments, deleting or inserting is of at least one nucleotide from or to the genome of the at least one cell of a crustacean embryo compared to a control non-edited genome.

In some embodiments, deleting or inserting is of a plurality of nucleotides from or to the genome of the at least one cell of a crustacean embryo compared to a non-edited control genome.

In some embodiments, a non-edited control genome is of a wildtype crustacean, an embryo thereof, or a cell thereof. In some embodiments, a non-edited control genome is of an embryo or a cell thereof before contacting according to the method of the invention. In some embodiments, the non-edited control genome is a genome of a crustacean, an embryo thereof, or a cell thereof. In some embodiments, a non-edited control genome is a reference genome or a genetic background genome. A non-edited control genome may be available online, e.g., Genbank or the like, as would be apparent to one of ordinary skill in the art.

As used herein, the term “plurality” refers to any integer being equal to or greater than 2.

In some embodiments, a plurality of nucleotides comprises 2 to 25, 2 to 20, 2 to 17, 2 to 15, 2 to 10, 5 to 20, 5 to 17, 5 to 15, 2 to 8, or 2 to 5. Each possibility represents a separate embodiment of the invention.

In some embodiments, deleting comprises removing, existing, or both, 2 to 25, 2 to 20, 2 to 17, 2 to 15, 2 to 10, 5 to 20, 5 to 17, 5 to 15, 2 to 8, or 2 to 5 nucleotides, from the edited genome of at least one cell compared to a non-edited control genome. In some embodiments, a genome edited according to the metho do the invention is devoid of 2 to 25, 2 to 20, 2 to 17, 2 to 15, 2 to 10, 5 to 20, 5 to 17, 5 to 15, 2 to 8, or 2 to 5 nucleotides being present in a non-edited control genome. Each possibility represents a separate embodiment of the invention.

In some embodiments, a non-edited control genome comprises 2 to 25, 2 to 20, 2 to 17, 2 to 15, 2 to 10, 5 to 20, 5 to 17, 5 to 15, 2 to 8, or 2 to 5 nucleotides being absent in a genome edited according to the method of the invention. Each possibility represents a separate embodiment of the invention.

In some embodiments, deleting comprises introducing a mutation in at least one gene of the edited genome. In some embodiments, the deleting results in introduction of a in at least one gene of the edited genome.

In some embodiments, the mutation comprises a non-synonymous mutation, a nonsense mutation, a frameshift, or any combination thereof, in at least one gene of the edited genome.

In some embodiments, the mutation renders the gene, a transcript transcribed therefrom, a protein product thereof, or any combination thereof, at least partially inactive. In some embodiments, the mutation renders the gene, a transcript transcribed therefrom, a protein product thereof, or any combination thereof, fully inactive. In some embodiments, the mutation is a pre-mature stop codon. In some embodiments, the mutation disrupts the protein product. In some embodiments, the protein product is a short version or is shorter than the protein product encoded by the same gene of the non-edited control genome.

In some embodiments, the gene is a gene of interest. In some embodiments, a gene of interest comprises any gene the manipulation of which is desired to enhance, improve, select, or any combination thereof, a phenotype of a crustacean. Such a gene of interest may be related to growth, survival, reproduction, disease resistance, or others.

In some embodiments, the embryo comprises a plurality of embryos.

In some embodiments, 10-100%, 10-90%, 10-80%, 10-70%, 10-60%, 10-50%, 10-40%, 10-30%, 10-20%, 20-100%, 20-80%, 30-60%, 30-80%, 40-100%, 40-70%, or 20-50%, of a plurality of embryos being contacted with a gene editing agent comprises at least one cell comprising an edited genome. Each possibility represents a separate embodiment of the invention.

In some embodiments, 50-100%, 50-90%, 50-80%, 50-70%, 50-60%, 60-100%, 70-100%, 80-100%, 90-100%, 60-90%, 60-80%, 70-80%, 70-90%, or 40-100%, of the plurality of embryos being contacted with a gene editing agent survive the contacting. Each possibility represents a separate embodiment of the invention.

In some embodiments, contacting comprises electroporating. In some embodiments, the method comprises mixing the at least one cell of a crustacean embryo with an effective amount of a gene editing agent (thereby obtaining a mixture comprising the at least one cell and the agent), and electroporating or applying voltage to the mixture of the at least one cell and agent. In some embodiments, electroporation comprises applying a voltage to a plurality of cells, such as a plurality of crustacean embryonic cells. In some embodiments, the method further comprises adjusting the osmolality of the mixture to about: 400 to 440 mOsm, 410 to 430 mOsm, or 415 to 435 mOsm. Each possibility represents a separate embodiment of the invention. In some embodiments, the at least one cell, agent, or both, are suspended in a medium characterized by osmolality of about: 400 to 440 mOsm, 410 to 430 mOsm, or 415 to 435 mOsm. Each possibility represents a separate embodiment of the invention. In some embodiments, the method comprises suspending and/or contacting the at least one cell in a medium characterized by osmolality of about: 400 to 440 mOsm, 410 to 430 mOsm, or 415 to 435 mOsm. Each possibility represents a separate embodiment of the invention.

In some embodiments, the method comprises suspending and/or dissolving the agent in a medium characterized by osmolality of about: 400 to 440 mOsm, 410 to 430 mOsm, or 415 to 435 mOsm. Each possibility represents a separate embodiment of the invention.

As used herein, the term “electroporating” refers to applying voltage. In some embodiments, the method comprises applying a first pulse, such as a “poring pulse”, followed by a second pulse, such as a “transfer pulse”. In some embodiments, the first pulse, the second pulse, or both, comprises a plurality of pulses, e.g., first pulses and second pulses.

In some embodiments, the voltage of the first pulse ranges from: 155 to 195 V, 160 to 195 V, 165 to 195 V, 170 to 195 V, 175 to 195 V, 180 to 195 V, 185 to 195 V, 155 to 190 V, 155 to 185 V, 155 to 180 V, 155 to 175 V, 155 to 170 V, 155 to 165 V, 155 to 160 V, 160 to 190 V, 165 to 185 V, or 170 to 180 V. Each possibility represents a separate embodiment of the invention.

In some embodiments, the voltage of the second pulse ranges from: 5 to 35 V, 5 to 30 V, 5 to 25 V, 5 to 20 V, 10 to 30 V, 10 to 25 V, 10 to 20 V, 15 to 25 V, or 15 to 20 V. Each possibility represents a separate embodiment of the invention.

In some embodiments, the voltage is applied as a pulse. In some embodiments the voltage is applied as a single pulse. In some embodiments, the voltage is applied as a plurality of pulse.

In some embodiments, the first pulse is applied for a period of about 2 to 5 msec, 2.5 to 4 msec, 3 to 4 msec, 3.5 to 4 msec, or 2 to 4 msec. Each possibility represents a separate embodiment of the invention.

In some embodiments, the interval between each pulse of a plurality of first pulses is of about 40 to 60 msec, 30 to 70 msec, or 20 to 80 msec. Each possibility represents a separate embodiment of the invention.

In some embodiments, the second pulse is applied for a period of about 40 to 60 msec, 45 to 60 msec, 50 to 60 msec, 45 to 55 msec, or 45 to 50 msec. Each possibility represents a separate embodiment of the invention.

In some embodiments, the interval between each pulse of a plurality of second pulses is of about 40 to 60 msec, 30 to 70 msec, or 20 to 80 msec. Each possibility represents a separate embodiment of the invention.

In some embodiments, the electroporating comprises applying to the cell at least one first pulse or a plurality thereof proceeded by at least one second pulse or a plurality thereof.

In some embodiments, the method further comprises a step comprising culturing a crustacean embryo comprising at least one genome-edited cell. In some embodiments, the culturing is to a developmental stage selected from: larva, post larva, juvenile, adult, or any combination thereof.

In some embodiments, a crustacean is a decapod crustacean. In some embodiments, a decapod belongs to the suborder Dendrobranchiate or Pleocyemata. In some embodiments, the decapod crustacean is a prawn. In some embodiments, the decapod crustacean is a shrimp. In some embodiments, the decapod crustacean belongs to the family Penaeidae. In some embodiments, the decapod crustacean belongs to the genus Penaeus. In some embodiments, a decapod crustacean belongs to the genus Macrobrachium. In some embodiments, a decapod crustacean belonging to the genus Macrobrachium is or comprises Macrobrachium rosenbergii. In some embodiments, a decapod crustacean is a Penaeid shrimp.

According to another aspect, there is provided a crustacean comprising at least one cell comprising an edited genome, obtained according to the method of the invention.

### General

The term “subject” as used herein refers to an animal, more particularly to non-human mammals and human organism. Non-human animal subjects may also include prenatal forms of animals, such as, e.g., embryos or fetuses. Non-limiting examples of non-human animals include: horse, cow, camel, goat, sheep, dog, cat, non-human primate, mouse, rat, rabbit, hamster, guinea pig, pig. In one embodiment, the subject is a human. Human subjects may also include fetuses. In one embodiment, a subject in need thereof is a subject afflicted with and/or at risk of being afflicted with a condition associated with increased cell proliferation, deubiquitination activity, or combination thereof.

Any concentration ranges, percentage range, or ratio range recited herein are to be understood to include concentrations, percentages or ratios of any integer within that range and fractions thereof, such as one tenth and one hundredth of an integer, unless otherwise indicated.

Any number range recited herein relating to any physical feature, such as polymer subunits, size or thickness, are to be understood to include any integer within the recited range, unless otherwise indicated.

As used herein, the terms “subject” or “individual” or “animal” or “patient” or “mammal,” refers to any subject, particularly a mammalian subject, for whom therapy is desired, for example, a human.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Additional objects, advantages, and novel features of the present invention will become apparent to one ordinarily skilled in the art upon examination of the following examples, which are not intended to be limiting. Additionally, each of the various embodiments and aspects of the present invention as delineated herein above and as claimed in the claims section below finds experimental support in the following examples.

### EXAMPLES

Generally, the nomenclature used herein, and the laboratory procedures utilized in the present invention include molecular, biochemical, bioengineering, bioprocessing, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); Molecular Cell Biology Berk A. et al. 8th edition; Molecular Biotechnology: Principles and Applications of Recombinant DN, Glick BR. 5th edition; Culture of Animal Cells: A Manual of Basic Technique and Specialized Applications Freshney IR, 7th edition; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization-A Laboratory Course Manual” CSHL Press (1996); all of which are incorporated by reference. Other general references are provided throughout this document.

### Materials and Methods

### Animals and Cells

All early developmental stage M. rosenbergii embryos used for injection and cell culture were acquired through the BGU breeding program, and a number of egg-carrying gravid females were obtained from Colors Farm Ltd (Israel). These gravid females were produced in a tank system as follows: A single blue claw male was housed with four to six mature females in a 500-L tank. Each tank was checked daily for the typical male reproductive guarding behavior as an indication of gravid females in the tank. Gravid females were then moved to a separate holding aquarium, and the embryos were examined under a light microscope to determine their stage, as described below. One- to four-cell embryos were used for the whole-animal editing platform, while embryos that had already passed the four-cell threshold were kept for 9-13 days up to the eyed-egg embryo stage and then used for the primary cell culture editing platform.

### Embryo Stage Identification

Embryos were delicately removed from the female with a fine forceps and examined under a light microscope (Nikon H550s, Nikon, Japan). The number of cells was determined by visual inspection. When visual confirmation of cell division was not adequate, cell division was shown by injecting the embryos with Fast Green FCF dye (Allied Chemicals, Thailand), which does not cross membranes, thereby enabling the visualization of cell divisions.

In-silico Identification of Mr-cofilin in the M. rosenbergii transcriptome

The term ‘cofilin’ was used as a search keyword in an in-house M. rosenbergii embryonic transcriptome library. Hits of Pfam analysis were taken for a blastx search until a match to the close crustacean species Litopenaeus vannamei (NCBI Reference Sequence: XP_027225677.1) was found. The expression pattern during embryonic development was then generated from the above transcriptome library. The transcript was aligned to an in-house M. rosenbergii genome to obtain the genomic structure. The genomic structure was then modeled in IBS software. Finally, the transcript was translated in silico using ExPASy (web.expasy.org/translate) to determine the open reading frames (ORFs).

### Embryo Preparation and Sterilization

To prevent infection during incubation, fertilized eggs were removed from the brood chamber with fine forceps (or a toothbrush) and placed in a 50-mL tubes, each containing sterile salt water (15 ppt Coral Pro Salt; RedSea, Israel) and 10 μL of methylene blue. Following washing for 10 min in this solution, the eggs were rinsed with sterile saltwater containing iodine (0.001 vol. %) for 1 min, with sterile saltwater containing formaldehyde (4%) for 5 min, and finally with sterile salt water for 5 min. The fertilized eggs were held in a small petri dish of salt water until injected.

### Embryo Injections

A ribonucleoprotein (RNP)-Cas9 complex was prepared by mixing 9.5 pmol of synthesized single-guide RNA (sgRNA; IDT, USA) with 5.6 pmol of Cas9 protein (IDT) in a total volume of 1.8 μL. The complex was incubated for 5 min at room temperature before mixing with 0.05% phenol red to a total volume of 4 μL. For Cas9 mRNA delivery, 1 μg/μL GeneArt™ CRISPR Nuclease mRNA (A29378, ThermoFisher) was mixed with similar concentration of gRNA as mentioned above. All injections into M. rosenbergii fertilized eggs were performed with IVF micropipettes (Origio, USA), using a pneumatic PicoPump PV830, a vacuum injection system (WPI, USA), and two manual micromanipulators (WPI-M3301R/L, WPI). Pressure was set at 20 psi, and the injection duration was set at 100 ms. By using a vacuum pump (Schwarzer Precision, Germany) at a negative pressure of 5 psi, eggs were kept in place for injection with a holding capillary. Then, the injection capillary was inserted into the egg, and successful insertion of the material was verified through the observation of a small phenol red stain at the moment of injection.

### Injection Volume Calculation

The PV830 injection system included a compressed hydrogen system to inject the RNP solution. The volume delivered per millisecond was calculated from the equations below using a known injection period of 500 ms followed by the measurement of the diameter of a drop of injected liquid on the tip of the injection capillary. The calculations below show that at an injection rate of 6.72 pL ms⁻¹, 100 ms of injection would deliver 0.67 nL.

$$\frac{Bubble volume ( \frac{4}{3} π {( {r}_{bubble radius} )}^{3} )}{{t}_{Injection time}} = Injection {rate}_{t} ( \frac{Injection volume}{{t}_{Injection time}} ) \\ micron cubed ( {μm}^{3} ) = pico Liter ( pL ) * 1 {0}^{- 3} \\ ( \frac{4}{3} π {( r )}^{3} ) * 1 {0}^{- 3} \frac{pL}{{μ m}^{3}} = Injection volume ( pL ) \\ t ( milliseconds ( ms ) ) = 500 ms \\ r ( avg ; n = 3 ) = \frac{diameter = 18 \; 5.8 \pm 2.9 μm}{2} = 92.9 \\ ( \frac{4}{3} {π ( 92.9 )}^{3} ) * 1 {0}^{- 3} \frac{pL}{{μm}^{3}} = 3359.9 pL \\ Injection {rate}_{t} = \frac{3359.9 pL}{500 ms} = 6.72 \frac{pL}{m s}$$

Embryo Care, DNA extraction, PCR Amplification and Amplicon Sequencing

Injected embryos were transferred to a sterile incubator in small petri dishes, 20-30 embryos per dish, in an aqueous salt solution (15 ppt Coral Pro Salt) and kept at 27° C. for one week. The incubation solution was changed daily, and the eggs were examined visually for dead or malformed embryos. If infection (mainly fungal) was observed, non-contaminated eggs were moved to fresh dishes and further closely monitored. After one week, embryos were separated into individual tubes, and DNA was extracted as follows. Briefly, eggs were submerged in NaOH (0.2 N) and heated to 70° C. for 20 min in a Thermocycler Life Eco (Falc). After rough pipetting to ensure lysis, 50 μL of Tris HCI (0.04 M) were added to neutralize the acidity. DNA was then used as a template for amplification by PCR with specific primers flanking the gRNA. PCR products were purified using EPPiC Fast (A&A Biotechnology, Poland) and sent to the Macrogen sequencing lab (Amsterdam, Netherlands) for Sanger sequencing.

### Embryonic Cell Extraction, Editing, and Editing Monitoring

Prior to the isolation of embryonic cells, M. rosenbergii egg-bearing females were disinfected in a methylene blue water bath (5 drops to 10 L water) at least one day prior to cell extraction. Eggs were further disinfected by washing for 10 min in crustacean physiological saline (CPS) plus antibiotics (PEN/STREP, Biological Industries, Israel) and 0.5 μg/mL of the antifungal preparation Voriconazole (Sigma) in a rotator. Thereafter, the eggs were strained off in a 100-μm strainer (SPL Life Sciences, Republic of Korea) and washed for 1 min in 4% formalin-CPS solution, 1 min in 0.01% iodophor-CPS solution, and finally 10 min in CPS. Eggs were transferred to sterile Eppendorf tubes, containing 200 μL of Halt™ Protease and Phosphatase Inhibitor Cocktail (PI; Thermo-scientific) and 300 μL of CPS and homogenized. The homogenate was filtered through a 100-μm strainer, and the cells that passed through the strainer were collected in a 3-mL petri dish. Thereafter, these cells were transferred to a 15-mL tube and centrifuged at 850 g for 6 min at 18° C. The supernatant was discarded, and the pellet was resuspended in 1 mL of PI-CPS and then transferred to a Percoll step gradient comprising 3 mL of 100% physiological Percoll (13.5 mL commercial Percoll with 1.5 mL of CPS×10), 2 mL of 50% Percoll (physiological Percoll diluted with CPS×1), 4 mL of 25% Percoll, and 3 mL of 12.5% Percoll. Cells were centrifuged at 850 g for 30 min at 18° C. Cell fractions were transferred to a 15-mL tube and washed once with CPS. The cells were seeded at a density of 3×10⁶ cells per well in a 6-well plate and grown in Opti-MEM Medium whose osmolality was adjusted to 420 mOsm with CPS. Plates were incubated at 28° C. with 5% CO₂ for 24 h before RNP nucleofection. To monitor editing success, PCR products, using the genomic DNA as template, were sent to the Technion-Israel Institute of Technology (Israel) for next generation sequencing (Miseq Run V2; 2×150 bp, assuming 4 M reads per ends per run). The indels caused by the repair of the double-strand breaks through nonhomologous end-joining (NHEJ) were monitored.

### Cell Nucleofection

Nucleofection was performed using an Amaxa P3 Primary Cell 16-well Nucleocuvette Strips kit (V4XP-3032; Lonza, Switzerland) and an Amaxa 4D-Nucleofecter (Lonza). The RNP Cas9 complex was formed by mixing 2.64 μL (163 pmol) of Cas9 62 μM (1081059; IDT) with 1.76 μL (176 pmol) of sgRNA 100 μM (IDT) in P3 nucleofection buffer (total volume of 11 μL); the mixture was incubated at room temperature for 20 min. For mRNA Cas9 delivery, GeneArt™ CRISPR Nuclease mRNA (A29378; ThermoFisher) was used in a final concentration of 0.06 μg/μL. An amount of 4×10⁵ primary cells were harvested, washed once in Opti-MEM, and resuspended in 28.6 μL of P3 nucleofection buffer. The cell suspension was mixed with RNP-Cas9, and 20 μL of the cell-RNP suspension was transferred to each of two wells of the 16-well strip nucleocuvette. Cells were electroporated using program CL-137 on the 4D-Nucleofector. Immediately after nucleofection, 80 μL of Opti-MEM+10% FBS was added to each well at room temperature, and all 100 μL of cell suspension were transferred to a 96-well plate containing 50 μL of pre-warmed medium. The plate was incubated at 28° C. under 5% CO₂. Edited cells were harvested three days after nucleofection. For DNA extraction, cells were suspended in culture medium and centrifuged at 11,000 g for 5 min. The supernatant was discarded, and the cells were resuspended in 10 μL of NaOH (0.2 N). Tubes were heated to 70° C. for 20 min and vortexed before the addition of 50 μL of Tris HCI (0.04 M).

### Phenotypic Proof of Concept

M. rosenbergii embryos start developing eyes approximately one week after fertilization, and therefore eye development was used in the present study as an early indicator of successful genomic KO. Zygote to second nuclear division embryos at 3-7 h post fertilization were taken from a gravid female and sterilized as described above. Three different guides were injected, Hox3 (n=44) and Hox4 (n=41), aimed at the Pax6 Hox motif as a mean of maximizing KO effectiveness, and Mr-cofilin (n=28) as a positive control for the editing. Non-injected embryos served as the negative control for the phenotype. Individual gRNA and primers are detailed in Table 1.

TABLE 1
Guide RNAs and primers for Sanger sequencing

| Sequence | 5′ to 3′ sequence | SEQ ID NO: |
|---|---|---|
| Hox3 gRNA | TCTTTGCAGCCAGTCTCTCG | 1 |
| Hox4 gRNA | TCTCGCGGGCAAATACGTCA | 2 |
| Hox3,4 forward primer | TTTCACTGTAATGATTTTTCAAGAGC | 3 |
| Hox3,4 reverse primer | CAGACAAATAACCTTCTAAGCAAGGG | 4 |
| Mr-cofilin gRNA | CATATGACTAGCTGGCCACA | 5 |
| Mr-cofilin forward primer | TATGGCCGAAGCATCTTATGAATGT | 6 |
| Mr-cofilin reverse primer | CACAACTCTTTCCACACACCAACAA | 7 |

After injections of 0.67 nL (for each embryo), the embryos were kept at 28° C. for 20 days up to hatching, with monitoring every two days as described above. Two hatchlings from each treatment were taken for Sanger sequencing, and the remaining hatchlings (n=3 for each group) were examined under a microscope (Nikon H550s, Nikon, Japan) for phenotypic outcomes in the developing eye. Eye area was calculated by measuring the length and width of the eye in perpendicular lines, then multiplying the measurements and halving the result

$$( Eye area = \frac{Eye length * Eye width}{2} ) .$$

### Sequencing and Statistical Analyses

Sequences from Sanger sequencing were analyzed by the Synthego ICE program (ice.synthego.com) and charted according to the KO score. Crispresso2 (crispresso.pinellolab.partners.org/submission) was used to determine the editing efficiency of various guides in the genomic editing experiments using the default settings of the website. Comparisons and statistical analyses were performed with Statistica v13.3 software (StatSoft, Ltd., USA), using one-way ANOVA, followed by a post hoc Tukey's HSD test with p≤0.05 regarded as a significant difference. All percentage data was converted to decimal form and transformed by applying the square-root of the tangent. To test the difference in the pattern of editing between injected embryos and cells in primary culture across a 40-bp window, two analyses were used: First, a non-parametric two-sample Kolmogorov-Smirnov test and a permutations test on the absolute difference in skewness between the two editing distribution patterns were used. Second, data from embryos and cells were pooled and then sampled without replacement to generate two random sample distributions. The absolute difference (two-sided test) in skewness between the two random sample distributions was calculated. This procedure was repeated 10,000 times. Permutation P values were calculated as the proportion of cases in which the absolute difference obtained during the random simulations was equal to or greater than that detected in the original data set. These analyses were performed using MATLAB version 2020a (The Math Works, Inc., 2020, USA).

### Example 1

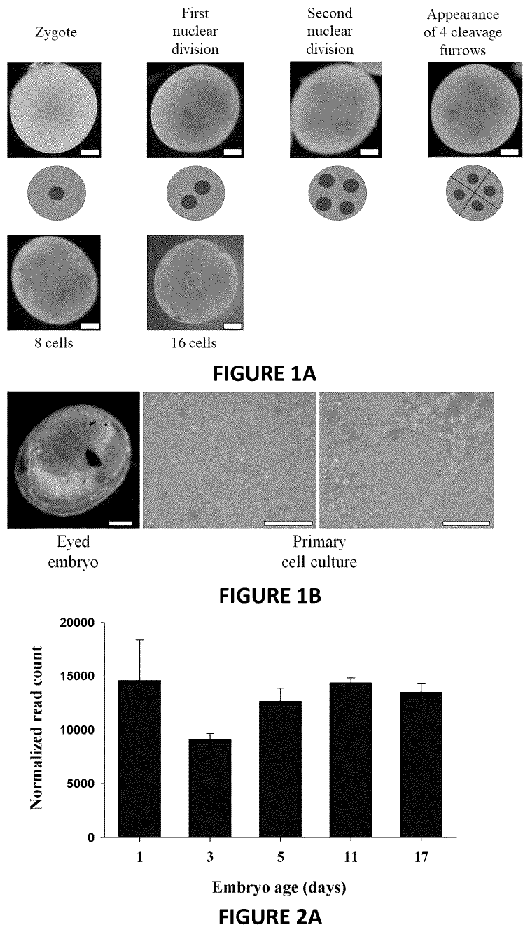

To tailor the genome editing method in M. rosenbergii, the inventors studied two time periods during embryonic development, namely, the period of early divisions of the embryonic cells that were used to construct the edited embryos and the eyed-embryo stage, 10-16 days after fertilization. Immediately after fertilization, upon visual inspection, the eggs appear bright yellow and entirely filled with yolk vesicles, while under a light microscope, a darker mass (the nucleus) can be seen at the center of the cell. At 3-4 HPF, the nucleus splits into two (first nuclear division), but no cell wall is visible (FIG. 1A, top). At 6 HPF, the two nuclei divide again, and only at 8 HPF are the first cellular divisions into four cells, each with a nucleus at the center, visible. The next two cell divisions are correlated with nuclear divisions, 9 HPF for the 8-cell stage, and 10.5 HPF for the 16-cell stage (FIG. 1A, bottom). When the eyes started to develop and became visible as black slits on the sides of the embryo (eyed-embryo stage, FIG. 1B, left), cells were extracted from the embryos and cultured for 24 h prior to electroporation and editing manipulations (FIG. 1B, middle and right). At this stage, a considerable quantity of cells could be extracted to establish a primary cell culture (FIG. 1B, right).

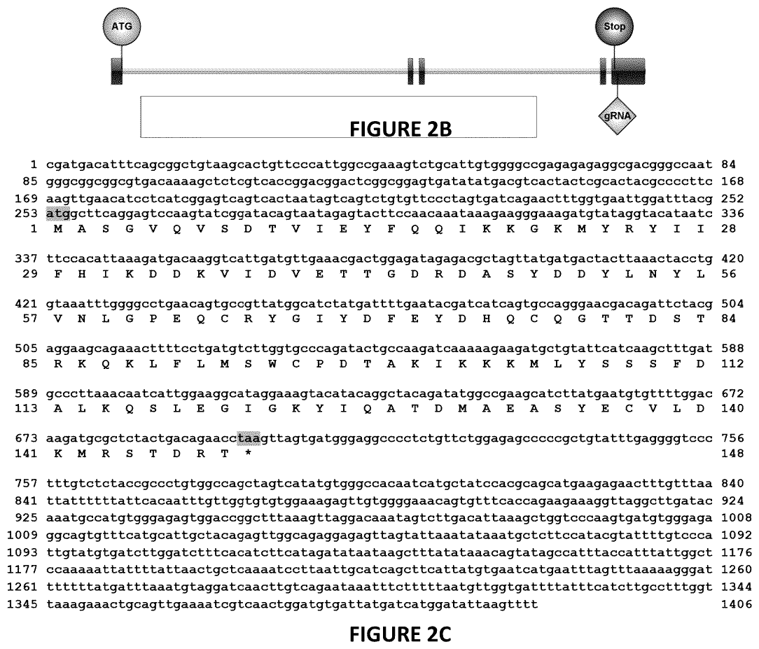

A key word search for “cofilin” in an in-house M. rosenbergii embryonic library revealed a transcript of 1061 nucleotides. This transcript was termed Mr-cofilin and was submitted in the GenBank® (accession number OL743530). A BLAST search of this transcript in the NCBI gave the highest similarity to a transcript of M. nipponense (Sequence ID: CP062035.1), with an E value of zero and 98.68% identity. This transcript was highly expressed in all embryonic stages that are represented in the M. rosenbergii embryonic library (days 1, 3, 5, 11 and 17; see FIG. 2A). A BLAST search in the genome of M. rosenbergii revealed that the genomic sequence of Mr-cofilin contains 5 exons, as shown in FIG. 2B. The sgRNA design for the Mr-cofilin gene was identified by the IDT online tool.

In an in-house M. rosenbergii embryonic library, Mr-cofilin showed a high relative expression throughout embryonic development (FIG. 2A), with day 1 showing a read count average±standard error of 14,604±3,779 and day 3, a slight drop to 9,076±601. The read counts for days 5, 11, and 17 were 12,682±1210, 14,358±479, and 13,531±770, respectively. As may be seen in FIG. 2B, the genomic structure of Mr-cofilin includes five exons and four introns, with two long introns spanning over 4,000 bp and two short introns of under 150 bp. The mature mRNA comprises 1,406 bp with an ORF of 447 bp and long 5′ and 3′ UTRs. The guide RNA selected for the present study is situated at the 3′ UTR at position 769 (FIG. 2C).

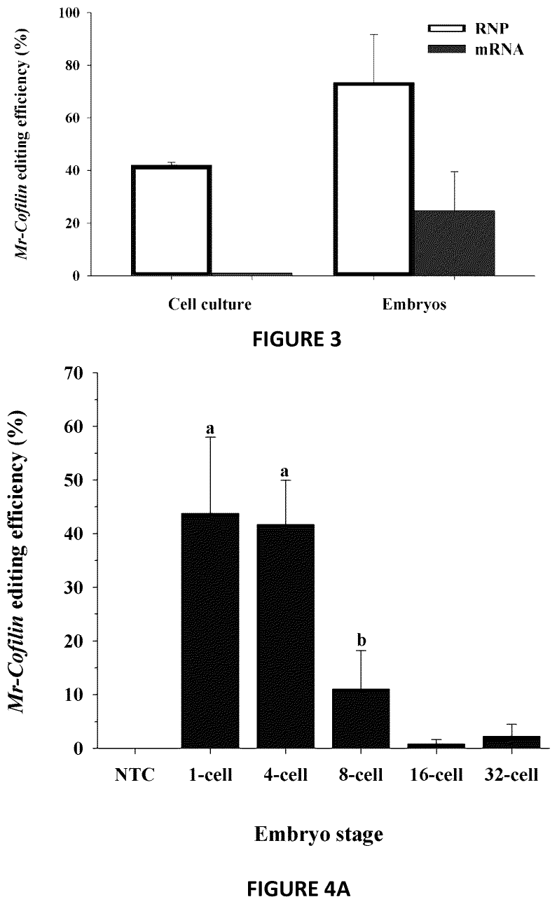

Mr-cofilin was found to be a highly expressed gene (FIG. 2A) whose editing did not cause fatalities; it was therefore used for the study of the optimal delivery method of Cas9 into the prawns. The current ICE results for experiments conducted in line with recommendations from the literature for the use of either Cas9 mRNA plus the guide or RNP showed that in M. rosenbergii mRNA injection into embryos was not as effective as RNP (FIG. 3), with an average of 24.7±14.8% successful editing for mRNA (n=5) versus 73.4±18.2% for RNP (n=6). The same trend was evident for the primary cell culture, with no detectable editing for mRNA versus 42±1% successful editing for RNP (n=8 for both).

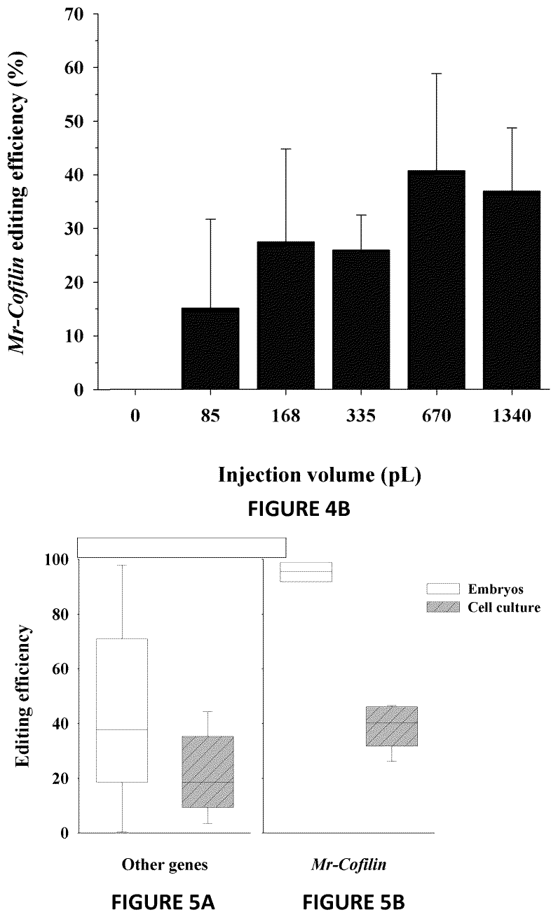

To determine the optimal schedule in terms of cell division for successful embryonic editing, embryos of different cell numbers, from fertilization up to the 32-cell stage, were injected with Cas9 RNPs (FIG. 4A). ICE results for the first two stages of no cellular divisions (1 cell) and the first division (4 cells) were similar, averaging 43.8±14.2% (n=5) and 41.6±8.3% (n=6), respectively. Only at the 8-cell stage was there a significant drop in editing success (p=0.02), with 11±7.2% (n=6) editing efficiency. The 16-cell stage appeared to be unaffected, with only a single case showing 5% editing efficiency, and the rest showed no editing (n=6). The same trend was evident for the 32-cell stage, with an average of 2.3±2.3% editing efficiency. Based on these results the inventors narrowed the effective injection period to embryos of one to four cells. Experimentation aimed to adjust the injection volume of Cas9 RNPs for 1-cell to 4-cell embryos yielded the following ICE results for six different volumes (doubling the volume in each step; FIG. 4B): 84 pL yielded 15.2±16.6 editing efficiency; 168 pL, 27.6±17.3%; 335 pL, 28.7±7.1%; and 670 pL 43.2±17.7%; a further increase in volume to 1.34 μL did not result in an increase in effectiveness, giving 30.3±12.4%. No significant difference was seen in embryo survival between the volumes checked.

Based on the above calibration results, 80 gRNAs targeting 23 different genes were tested. Percentage editing efficiency was calculated and showed that guide effectiveness varied in both edited embryos and embryonic cells in culture (FIG. 5). The rate of success in injected embryos ranged from no editing (0%) to 100% editing efficiency (n=40). In contrast, primary cell culture showed a narrower range, with editing efficiency ranging from 0% to a maximum of 64.3% (n=69). Mr-cofilin independent repeats were constant, averaging at 95.4±2.7% (n=3) for injected embryos versus 38.7±3.4% (n=7) for primary cell culture.

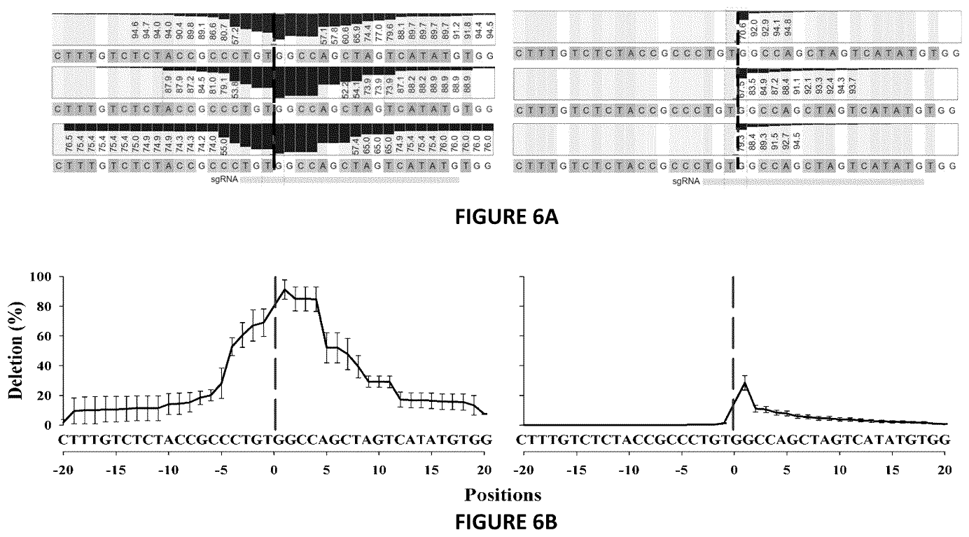

Crispresso 2 visual representation showed a striking difference between the editing patterns of the embryo and the embryonic cells in culture. To depict this phenomenon graphically, FIG. 6A shows three representative independent Mr-cofilin injections into embryos resulting in similar deletion patterns, with most deletions missing the first base upstream of the cut site and gradually decreasing further away to each side of the cut site. In contrast, embryonic cells in primary cell culture (FIG. 6B) showed a different editing pattern, with most deletions being in the same base, one upstream of the cut site with a positively skewed pattern towards the 3′ end of the gRNA showing a gradual drop to 0. These differences in patterns between embryos and cells were consistent with the patterns obtained using other guides targeting other genes. The above difference in the pattern of editing between embryos and cells was found to be statistically significant using the non-parametric two-sample Kolmogorov-Smirnov test (p<0.0001) and a permutations test on the absolute difference in skewness between the two patterns across a 40-bp window (p<0.001).

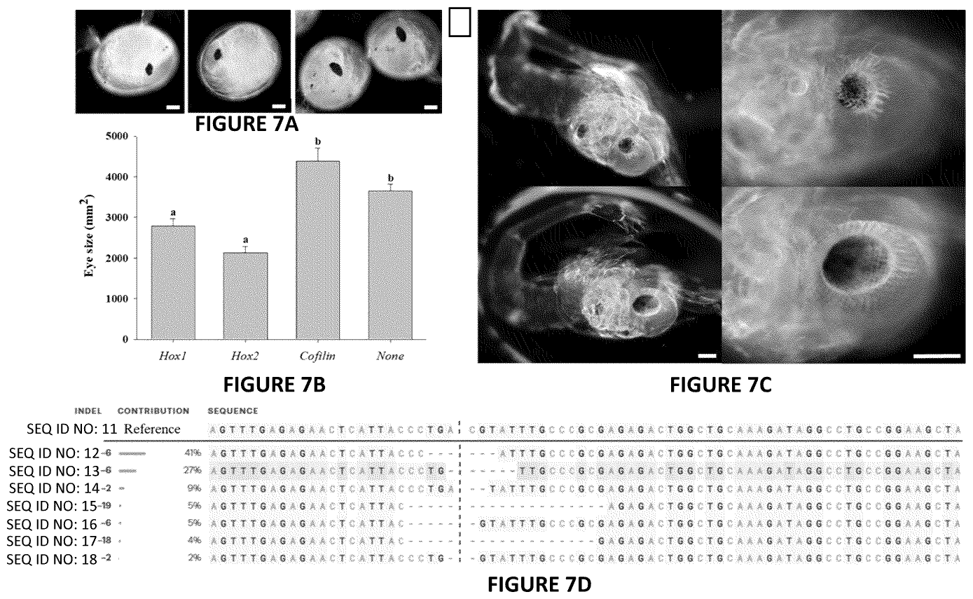

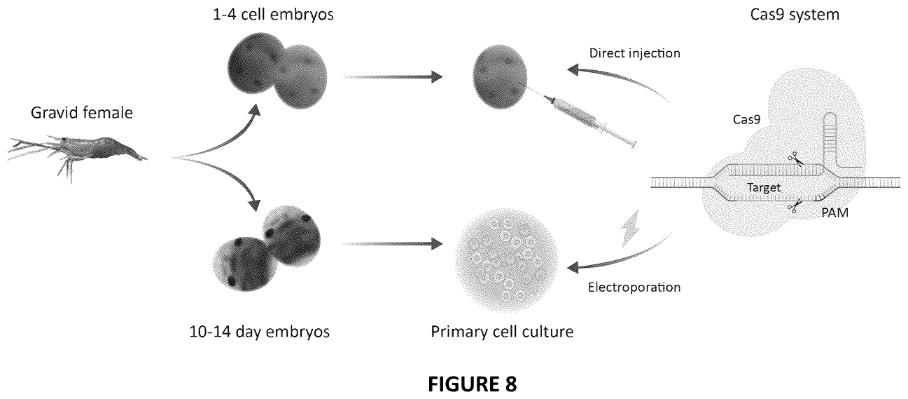

Phenotypic proof of concept was provided by investigating the effects of KO on eye development in embryos, which later hatched into larvae. Initial comparisons of eye size at 15 days post injection (FIG. 7A) showed a significant difference (p<0.001) between Hox3,4-injected embryos on the one hand and Mr-cofilin-injected and non-injected embryos on the other. Average eye size was 2,126±164 mm² for Hox4-injected embryos (n=6) and 2,789±183 mm² for Hox3-injected embryos (n=6); whereas significantly higher values were obtained for Mr-cofilin-injected and wild-type embryos, namely, 4,382±321 mm² (n=5) and 3,647±167 mm² (n=5), respectively (FIG. 7B). Survival rates of the injected embryos averaged 15%, with 7 of 44 and 5 of 41 hatching into larvae in Hox3-and Hox4-injected embryos, respectively. Hatched larvae with Pax6 KO exhibited smaller eyes than the controls, together with a deformity of the crystalline cones (FIG. 7C). Sanger sequencing confirmed that the Pax6 KO affected 100% of the cells (FIG. 7D).

### Example 2

The inventors have further tested the examined the effects of pulse voltage and pulse length (msec) on genome editing (reflected as transfer efficiency (TE)) and viability of primary cell cultures of the giant freshwater prawn M. rosenbergii upon electroporation.

For electroporation, 600,000 cells per reaction were suspend in 60 μl of Opti-MEMTM Medium (Gibco. Thermo Fisher Scientific). The osmolality of the medium was adjusted to 420 mOsm using crustacean physiological saline. For RNP preparation, sgRNA (100 μM) and Cas9 (62 μM) were diluted with Opti-MEM™ Medium to a final concentration of 3.72 μM (sgRNA) and 1.55 μM (Cas9) in a total volume of 10 μl. The cells and RNP were mixed (a total of 70 μl) and were dispensed into a 1 mm gap cuvette. Electroporation was performed using the Super electroporator NEPA21 type II. Poring pulse as well as results, including viability (V) and transfer efficiency (TE) are presented in Table 2, hereinbelow. Further to the poring pulse, transfer pulse was uniformly applied in all different combinations of the poring pulse phase. Briefly, the transfer pulse program including voltage of 20 V; 50 msec pulse length; 50 msec pulse interval; repeated 5 times.

TABLE 2
Experimental parameters and results summary

| | Poring Pulse Parameters | | | | |
|---|---|---|---|---|---|
| # | Voltage (V) | Length (ms) | Interval (ms) | V (%) | TE (%) |
| 1 | Control (cells + DNA; w/o electroporation) | | | | |
| 2 | 100 | 4 | 50 | 69 | |
| 3 | 125 | 3 | 50 | 100 | 6% |
| 4 | 125 | 3.5 | 50 | 67 | |
| 5 | 150 | 3.5 | 50 | 36 | |
| 6 | 175 | 3.5 | 50 | 53 | 40% |
| 7 | 200 | 3 | 50 | <20 | |
| 8 | 225 | 2.5 | 50 | 23 | 19% |
| 9 | 275 | 0.5 | 50 | <20 | |
| 10 | 275 | 1 | 50 | <20 | |
| 11 | 275 | 1.5 | 50 | <20 | |
| 12 | 275 | 2.5 | 50 | <20 | |

In an initial examination, cell viability was assessed upon electroporation, the results showed that treatments #2, 3, and 4 had the highest viability rate, standing at 69%, 100%, and 67%, respectively. Treatments #5, 6, and 8, presented a moderate viability rate of 36%, 53%, and 23%, respectively. The remaining treatments were characterized by a rather low viability rate of below 20%.

Further, the inventors examined the rate of gene transfer efficiency (e.g., gene editing event(s)). Therefore, treatments #3, 6, and 8 were repeated in the presence of the RNP, including sgRNA and Cas9 nuclease. TE was determined by Sanger sequencing. While treatment #3 was characterized by the highest viability, but with an extremely low TE value of 6% TE. Treatment #8 was characterized by poor viability of 23% and TE values standing at 6-19%. Surprisingly, treatment #6 was characterized by a viability rate of more than 50% and TE value of 40%.

Therefore, the inventors conclude that a method for modifying or editing the genome of a crustacean embryonic cell requiring 50% survival rate and about 40% editing efficiency (or more) requires contacting the cells with a pulse of about 175 V applied for a period of about 3.5 msec. Further, the suitable resistance value of the electroporation should range between 0.025 and 0.06 KΩ. Osmolality of the medium in which the cells were suspended was about 420 mOsm.

Further to the above, the pressure applied by the injection system had to be adjusted in depth so as to successfully introduce an effective amount of an exogenous material, e.g., the gene editing agent according to the method of the invention, while maintaining embryo survival, wellbeing, and development. Application of injection pressure of 20 psi or below, resulted in high and relatively uniform survival rates. In sharp contrast, injection pressure of above 25 psi, such as 30 psi, severely reduced embryo survival, with the injected embryos bursting, e.g., yolk leaking out of the injected embryo, upon or during the injection.

In order to reduce human error while the needle being within the embryo, and thus reduce survival rates, it was decided to inject embryos at a pressure of about 20 psi, as lower pressures require prolonged periods to introduce a predetermined amount of an exogenous material compared to higher pressures, and pressure of 15-25 psi provide both suitable pressure to introduce and effective amount of the exogenous materials while preserving or mainlining survival rate. Further, during injection, a counter pressure of about 1 psi was found to be effective in prevention of water or yolk from the embryo entering/contaminating the needle (which is clearly deleterious to a high throughout process of multiple injections).

All publications, patents and patent applications mentioned in this specification are herein incorporated in their entirety by references into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method for editing the genome of at least one cell of a crustacean embryo, the method comprising contacting a crustacean embryo at the 1-cell to 8 cell stage with an effective amount of a gene editing agent, thereby editing the genome of at least one cell of a crustacean embryo.

2. The method of claim 1, wherein said crustacean embryo is at the 1-cell to 4-cell stage.

3. The method of claim 1, wherein said contacting is injecting.

4. The method of claim 3, wherein said injecting is pneumatically injecting.

5. The method of claim 4, wherein said pneumatically injecting is under pressure of 15 to 25 psi.

6. The method of claim 4, wherein said pneumatically injecting is for a period of 150-250 msec.

7. The method of claim 1, wherein said agent is a clustered regularly interspaced short palindromic repeat (CRISPR) system.

8. The method of claim 7, wherein said CRISPR system comprises: (i) a CRISPR-associated endonuclease 9 (Cas9) protein or a nucleic acid sequence encoding thereof; and (ii) a guide RNA (gRNA) targeting at least one protospacer within the genome of said at least one cell of a crustacean embryo.

9. The method of claim 7, wherein said CRISPR system comprises: (i) a Cas9 protein;
and (ii) a gRNA targeting at least one protospacer within the genome of said at least one cell of a crustacean embryo.

10. The method of claim 8, further comprising a step before said contacting, comprising mixing said Cas9 protein and said gRNA.

11. The method of claim 1, wherein said editing comprises deleting at least one nucleotide from the genome of said at least one cell of a crustacean embryo compared to a non-edited control genome.

12. The method of claim 11, wherein said deleting comprises introducing any one of a non-synonymous mutation, a nonsense mutation, and a frameshift, in at least one gene of said edited genome such that a transcript transcribed therefrom, a protein product thereof, or both, is at least partially inactivated.

13. The method of claim 1, wherein said embryo comprises a plurality of embryos, and wherein 10-100% of said plurality of embryos being contacted with said gene editing agent comprises at least one cell comprising an edited genome.

14. The method of claim 13, wherein 20-80% of said plurality of embryos being contacted with said gene editing agent comprises at least one cell comprising an edited genome.

15. The method of claim 13, wherein 50-100% of said plurality of embryos being contacted with said gene editing agent survive said contacting.

16. The method of claim 13, wherein 80-100% of said plurality of embryos being contacted with said gene editing agent survive said contacting.

17. The method of claim 1, further comprising a step comprising culturing said crustacean embryo comprising said at least one genome edited cell to a developmental stage selected from the group consisting of: larva, post larva, juvenile, adult, and any combination thereof.

18. The method of claim 1, wherein said crustacean is a decapod crustacean.

19. The method of claim 18, wherein said decapod crustacean is Macrobrachium rosenbergii.

20. A crustacean comprising at least one cell comprising an edited genome, obtained according to the method of claim 1.
